(19) United States
(12) Patent Application Publication
(10) Pub. No.: US 2019/0015421 A1
(43) Pub. Date: Jan. 17, 2019

(54) Biomarkers for Treating Cancer with Apilimod

(71) Applicant: LAM Therapeutics, Inc., Guilford, CT (US)

(72) Inventors: Sophia Gayle, East Haven, CT (US); Neil Beeharry, Guilford, CT (US); Sean Landrette, Meriden, CT (US); Chris Conrad, Guilford, CT (US); Paul Beckett, Yorktown Heights, NY (US); Marylens Hernandez, Guilford, CT (US); Tian Xu, Cambridge, MA (US); Jonathan M. Rothberg, Guilford, CT (US); Henri Lichenstein, Guilford, CT (US)

(21) Appl. No.: 16/070,950

(22) PCT Filed: Jan. 20, 2017
(86) PCT No.: PCT/US2017/014308

§ 371 Date: Jul. 18, 2018

Related U.S. Application Data
(60) Provisional application No. 62/281,341, filed on Jan. 21, 2016.

(51) Int. Cl. A61K 31/5377; A61P 35/00

(52) U.S. Cl. CPC A61K 31/5377; A61K 45/06; A61P 35/00

(57) ABSTRACT

The present disclosure relates to compositions and methods for treating cancer in a subject having cancer cells over-expressing a microphthalmia (MiT) transcription factor with apilimod and related compositions and methods.

### RELATED APPLICATION

This application claims priority from U.S. Provisional Patent Application Ser. No. 62/281,341, filed Jan. 21, 2016, the contents of which are hereby fully incorporated by reference.

### FIELD OF THE DISCLOSURE

The present disclosure relates to compositions and methods of using apilimod in the treatment of cancer.

### BACKGROUND OF THE DISCLOSURE

Apilimod, also referred to as STA-5326, hereinafter “apilimod”, is recognized as a potent transcriptional inhibitor of IL-12 and IL-23. See e.g., Wada et al. Blood 109 (2007): 1156-1164. IL-12 and IL-23 are inflammatory cytokines normally produced by immune cells, such as B-cells and macrophages, in response to antigenic stimulation. Autoimmune disorders and other disorders characterized by chronic inflammation are characterized in part by inappropriate production of these cytokines. In immune cells, the selective inhibition of IL-12/IL-23 transcription by apilimod was recently shown to be mediated by apilimod's direct binding to phosphatidylinositol-3-phosphate 5-kinase (PIKfyve). See, e.g., Cai et al. Chemistry and Biol. 20 (2013):912-921. PIKfyve plays a role in Toll-like receptor signaling, which is important in innate immunity.

Based upon its activity as an immunomodulatory agent and a specific inhibitor of IL-12/IL-23, apilimod has been proposed as useful in treating autoimmune and inflammatory diseases and disorders. See e.g., U.S. Pat. Nos. 6,858,606 and 6,660,733 (describing a family of pyrimidine compounds, including apilimod, purportedly useful for treating diseases and disorders characterized by IL-12 or IL-23 overproduction, such as rheumatoid arthritis, sepsis, Crohn's disease, multiple sclerosis, psoriasis, or insulin dependent diabetes mellitus). Similarly, apilimod was suggested to be useful for treating certain cancers based upon its activity to inhibit c-Rel or IL-12/23, particularly in cancers where these cytokines were believed to play a role in promoting aberrant cell proliferation role. See e.g., WO 2006/128129 and Baird et al., Frontiers in Oncology 3:1 (2013, respectively).

Each of three clinical trials of apilimod has focused on its potential efficacy in autoimmune and inflammatory diseases. The trials were conducted in patients having psoriasis, rheumatoid arthritis, and Crohn's disease. An open label clinical study in patients with psoriasis concluded that oral administration of apilimod showed immunomodulatory activity supporting the inhibition of IL-12/IL-23 synthesis for the treatment of TH1- and TH17-mediated inflammatory diseases. Wada et al., PLosOne 7:e35069 (April 2012). But the results of controlled trials in rheumatoid arthritis and Crohn's disease did not support the notion that IL-12/IL-23 inhibition by apilimod translates into clinical improvement in either of these indications. In a randomized, double-blind, placebo-controlled Phase TT clinical trial of apilimod in patients with rheumatoid arthritis, apilimod failed to alter synovial IL-12 and IL-23 expression. Krauz et al., Arthritis & Rheumatism 64:1750-1755 (2012). The authors concluded that the “results do not support the notion the IL-12/IL-23 inhibition by apilimod is able to induce robust clinical improvement in RA.” Similarly, a randomized, double-blind, placebo-controlled trial of apilimod for treatment of active Crohn's disease concluded that, although well tolerated, apilimod did not demonstrate efficacy over placebo. Sands et al Inflamm Bowel Dis. 2010 July; 16(7):1209-18.

The mammalian target of rapamycin (mTOR) pathway is an important cellular signaling pathway that is involved in multiple physiological functions, including cell growth, cell proliferation, metabolism, protein synthesis, and autophagy (La Plante et al Cell 2012, (149 (2), pp. 274-293). mTOR is a kinase that integrates intracellular and extracellular cues that signal the levels of amino acids, stress, oxygen, energy, and growth factors and regulates the cellular response to these environment cues. mTOR deregulation has been implicated in a wide range of disorders and diseases, including cancer, obesity, diabetes, and neurodegeneration. Certain components of the mTOR pathway have been explored as drug targets for treating some of these diseases. However, therapeutic efficacy has been limited, for example, in the treatment of some cancers, and some mTOR inhibitors have been shown to have an adverse effect on metabolism. The tuberous sclerosis complex tumor suppressor genes, TSC1 and TSC2, are negative regulators of mTOR.

### SUMMARY OF THE DISCLOSURE

We have previously shown that apilimod is a highly cytotoxic agent in TSC null cells, where the mTOR pathway is constitutively active. See WO 2015/112888, incorporated herein by reference in its entirety. We extended our findings to show that many cancer cell lines are sensitive to apilimod-induced cytotoxicity. Although B-cell lymphomas were the most sensitive to apilimod, that sensitivity unexpectedly did not correlate with c-Rel expression, IL-12 expression, or IL-23 expression. This was unexpected because earlier work had suggested apilimod would be useful against cancers where c-Rcl and/or IL-12/23 expression were critical in promoting aberrant cell proliferation. In further work we showed that instead, apilimod's cytotoxicity arose from its inhibition of intracellular trafficking which resulted in increased apoptosis. This activity could not have been predicted based upon apilimod's immunomodulatory activity via its inhibition of IL-12/23 production.

The present disclosure is based in part on the surprising discovery that the transcription factor TFEB enhances sensitivity to apilimod. TFEB is a member of the microphthalmia (MiT) transcription factor family and thus it follows that cancers identified as having high levels of one or more MiT transcription factors are good candidates for treatment with apilimod. Accordingly, the present disclosure provides methods for identifying cancers that are susceptible to apilimod, the methods comprising assaying a sample of cancer cells from the cancer for overexpression of one or more MiT transcription factors. In embodiments, the MiT transcription factors are selected from TFEB, TFE3, TFEC, and MITF. In embodiments, the MiT transcription factor is selected from TFEB and TFE3, or both.

In one aspect, the disclosure also provides a composition for treating cancer in a subject having cancer cells overexpressing one or more MiT transcription factors, the composition comprising a therapeutically effective amount of apilimod, or a pharmaceutically acceptable salt thereof. In embodiments, the apilimod is apilimod dimesylate. In embodiments, the composition is in a form suitable for oral or intravenous administration. In embodiments, the composition further comprises at least one additional active agent, which may be selected from a therapeutic agent or a non-therapeutic agent, or a combination of a therapeutic agent and a non-therapeutic agent. In embodiments, the at least one additional active agent is a therapeutic agent selected from the group consisting of a protein kinase inhibitor, a platinum based anti-neoplastic agent, a topoisomerase inhibitor, a nucleoside metabolic inhibitor, an alkylating agent, an intercalating agent, a tubulin binding agent, and combinations thereof. In embodiments, the therapeutic agent is a protein kinase inhibitor. In embodiments, the protein kinase inhibitor is pazopanib or sorafenib, or a combination thereof. The composition may further comprise a non-therapeutic agent selected to ameliorate one or more side effects of the apilimod. In embodiments, the non-therapeutic agent is selected from the group consisting of ondanestron, granisetron, dolsetron, and palonosetron. In embodiments, the non-therapeutic agent is selected from the group consisting of pindolol and risperidone.

In embodiments, the cancer being treated is refractory to standard treatment or is metastatic.

In embodiments, the cancer is selected from a non-Hodgkins B cell lymphoma, a renal cell carcinoma, a melanoma, a clear cell sarcoma, an alveolar soft part sarcoma, or a perivascular epitheloid cell tumor. In embodiments, the cancer is a renal cancer. In embodiments, the renal cancer is selected from clear cell renal carcinoma, a transitional cell carcinoma, Wilms tumor (nephroblastoma), renal sarcoma, and benign (non-cancerous) kidney tumors, renal adenoma, oncocytoma, and angiomyolipoma. In embodiments, the renal cell carcinoma is selected from the group consisting of a papillary type I or type II, a chromophobe, a hybrid, an oncocytoma, a translocation, an angiomyolipoma, an oncocytic, a medullary, and a collecting duct carcinoma. In embodiments, the renal cancer contains a TFEB translocation. In embodiments, the TFEB translocation is a t(6;11) (p21; q12) translocation. In embodiments, the renal cancer has a mutation in the von Hippel-Lindau (VHL) gene.

In one aspect, the disclosure provides a method for treating cancer in a subject having cancer cells overexpressing one or more MiT transcription factors, the method comprising administering to the subject a therapeutically effective amount of apilimod, or a composition comprising apilimod, wherein the apilimod is apilimod itself (i.e., apilimod free base), or a pharmaceutically acceptable salt, solvate, clathrate, hydrate, polymorph, prodrug, analog or derivative thereof. In one embodiment, the apilimod is apilimod free base or apilimod dimesylate. In embodiments, the method further comprises a pretreatment step of assaying for the expression of the one or more MiT transcription factors in a biological sample from the subject, the biological sample containing cancer cells. The MiT transcription factors may be selected from TFEB, TFE3, TFEC, and MITF. In embodiments, the MiT transcription factor is selected from TFEB and TFE3, or both.

In embodiments, the method further comprises administering at least one additional active agent to the subject. The at least one additional active agent may be a therapeutic agent or a non-therapeutic agent. The at least one additional active agent may be administered in a single dosage form with the apilimod, or in a separate dosage form from the apilimod. In embodiments, the at least one additional active agent is a therapeutic agent selected from the group consisting of a protein kinase inhibitor, a platinum based anti-neoplastic agent, a topoisomerase inhibitor, a nucleoside metabolic inhibitor, an alkylating agent, an intercalating agent, a tubulin binding agent, PD-1/PDL-1 pathway inhibitor, and combinations thereof. In embodiments, the therapeutic agent is a protein kinase inhibitor. In embodiments, the protein kinase inhibitor is pazopanib or sorafenib, or a combination thereof. In embodiments, the at least one additional active agent is a therapeutic agent selected from the group consisting of sorafenib (Nexavar®), sunitinib (Sutent®) temsirolimus (Torisel®), everolimus (Afinitor®), bevacizumab (Avastin®), pazopanib (Votrient®), axitinib (Inlya®) and combinations thereof. In embodiments, the therapeutic agent is a PD-1/PDL-1 pathway inhibitor. In embodiments, the PD-1/PDL-1 pathway inhibitor is selected from pembrolizumab (Keytruda), avelumab, atezolizumab (MPDL3280A), nivolumab (BMS-936558), pidilizumab (CT-011), MSB0010718C, and MEDI4736.

In embodiments, the at least one active agent is a non-therapeutic agent selected to ameliorate one or more side effects of apilimod. In embodiments, the non-therapeutic agent is selected from the group consisting of ondanestron, granisetron, dolsetron, and palonosetron. In one embodiment, the non-therapeutic agent is selected from the group consisting of pindolol and risperidone. In one embodiment, the dosage form of the apilimod composition is an oral dosage form. In another embodiment, the dosage form of the apilimod composition is suitable for intravenous administration, administration is by a single injection or by a drip bag.

In one embodiment, the subject is a human cancer patient. In one embodiment, the human cancer patient in need of treatment with apilimod is on whose cancer is refractory to a standard chemotherapy regimen. In one embodiment, the human cancer patient in need of the treatment with apilimod is one whose cancer as recurred following treatment with a standard chemotherapy regimen. In one embodiment, the cancer is a renal cancer. In one embodiment, the renal cancer is a transitional cell carcinoma, Wilms tumor (nephroblastoma), renal sarcoma, and benign (non-cancerous) kidney tumors, renal adenoma, oncocytoma, and angiomyolipoma. In one embodiment, the renal cancer is a clear cell renal cell carcinoma.

In one embodiment, the standard chemotherapy regimen comprises one or more therapeutic agents selected from the group consisting of ibrutinib, rituximab, doxorubicin, prednisolone, vincristine, velcade, cyclophosphamide, dexamethasone and everolimus.

In one embodiment, the method is a method for treating renal cancer using a combination therapy comprising apilimod and a chemotherapy regimen for the treatment of the renal cancer. In embodiments, the chemotherapy regimen comprises a PD-1/PDL-1 pathway inhibitor. In embodiments, the PD-1/PDL-1 pathway inhibitor is selected from pembrolizumab (Keytruda, MK-3475), avelumab, atezolizumab (MPDL3280A), nivolumab (BMS-936558), pidilizumab (CT-011), MSB0010718C. and MEDI4736.

In another embodiment, the method is a method for treating renal cancer using a combination therapy comprising apilimod and an immunotherapy regimen for the treatment of the renal cancer. In one embodiment the immunotherapy regime is the Interleukin-2 (IL-2) regime or the alpha-interferon regime. In one embodiment, the immunotherapy regimen comprises a PD-1/PDL-1 pathway inhibitor. In embodiments, the PD-1/PDL-1 pathway inhibitor is selected from pembrolizumab (Keytruda, MK-3475), avelumab, atezolizumab (MPDL3280A), nivolumab (BMS-936558), pidilizumab (CT-011), MSB0010718C, and MEDI4736.

In some embodiments, the method is a method for treating renal cancer using a combination therapy comprising apilimod and a protein kinase inhibitor regimen for the treatment of the renal cancer. In one embodiment the protein kinase inhibitor regimen is sorafenib, sunitinib, bevacizumab, lenvatinib, everolimus.

### BRIEF DESCRIPTION OF THE DRAWINGS

FIG. 1: heatmap representation of gene expression changes in SU-DHL-10 and WSU-DLCL2 B-NHL lines upon apilimod treatment. Red color represents up-regulated genes while blue color represents down-regulated genes.

FIG. 2: gene Ontology analysis of commonly up-regulated genes from FIG. 1 reveals and enrichment for lysosomal associated genes.

FIG. 3: LysoTracker staining in SU-DHL-6 and SU-DHL-10 for 24 and 48 hrs after treatment with 200 nM (blue) apilimod compared to DMSO treated control (red).

FIG. 4: nuclear and cytoplasmic levels of TFEB protein assayed by immunoblotting in SU-DHL-6 cells treated with apilimod (63 nM) for 2 hr.

FIG. 5: box plots showing relative TFEB mRNA levels across tumor types, extracted from CCLE (Barretina et al., 2012) with gene centric robust multi-array analysis-normalized mRNA expression data.

FIG. 6: stable CA46 (TFEB-deficient B-NHL) pools over-expressing either GFP control or TFEB were treated with 10-point apilimod dose response for 72 hrs.

FIG. 7A: sensitivity of different cancer cell lines to apilimod in a 5 day assay.

FIG. 7B: example dose response of renal cell line RCC-ER and normal colon cell line CCD841CoN to apilimod in a 5 day assay.

FIG. 8: anti-proliferative activity of apilimod versus standard of care drugs in clear cell RCC cell lines (n=5) in 5 day assays. *Values denote the geometric mean.

### DETAILED DESCRIPTION OF THE DISCLOSURE

The present disclosure provides compositions and methods related to the use of apilimod for treating cancer in a subject, preferably a human subject, in need of such treatment. The present disclosure generally relates to the use of apilimod to treat cancers characterized by overexpression of one or more MiT transcription factors, such as TFEB, TFE3, TFEC, and MITF, which are shown herein to be particularly sensitive to apilimod-induced cytotoxicity. Non-limiting examples of cancers that can be characterized as overexpressing an MiT transcription factor include non-Hodgkins B cell lymphomas, renal cell carcinomas, melanomas, clear cell sarcomas, alveolar soft part sarcomas, and perivascular epitheloid cell tumors. The disclosure also provides methods for identifying a cancer as sensitive to apilimod, the methods comprising assaying for the expression of one or more MiT transcription factors selected from TFEB, TFE3, TFEC, and MITF.

In addition, the present disclosure provides novel therapeutic approaches to cancer treatment based upon combination therapy utilizing apilimod and at least one additional therapeutic agent. The combination therapies described herein exploit the unique cytotoxic activity of apilimod which is shown to provide a synergistic effect when combined with other anti-cancer agents.

As used herein, the term “apilimod” may refer to apilimod itself (i.e., apilimod free base), or may encompass pharmaceutically acceptable salts, solvates, clathrates, hydrates, polymorphs, metabolites, prodrugs, analogs or derivatives of apilimod, as described below. The structure of apilimod is shown in Formula I:

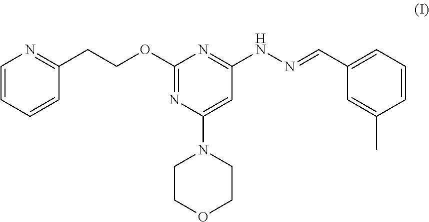 [SMILES: Cc1cccc(/C=N/Nc2cc(N3CCOCC3)nc(OCCc3ccccn3)n2)c1]

The chemical name of apilimod is 2-[2-Pyridin-2-yl)-ethoxy]-4-N′-(3-methyl-benzilidene)-hydrazino]-6-(morpholin-4-yl)-pyrimidine (IUPAC name: (E)-4-(6-(2-(3-methylbenzylidene)hydrazinyl)-2-(2-(pyridin-2-yl)ethoxy)pyrimidin-4-yl)morpholine), and the CAS number is 541550-19-0.

Apilimod can be prepared, for example, according to the methods described in U.S. Pat. Nos. 7,923,557, and 7,863,270, and WO 2006/128129.

As used herein, the term “pharmaceutically acceptable salt,” is a salt formed from, for example, an acid and a basic group of apilimod. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, besylate, gentisinate, fumarate, gluconate, glucuronate, saccharate, formate, benzoate, glutamate, methanesulfonate, ethanesulfonate, benzenesulfonate, p-toluenesulfonate, and pamoate (e.g., 1,1*-methylene-bis-(2-hydroxy-3-naphthoate)) salts.

The term “pharmaceutically acceptable salt” also refers to a salt prepared from an apilimod composition having an acidic functional group, such as a carboxylic acid functional group, and a pharmaceutically acceptable inorganic or organic base.

The term “pharmaceutically acceptable salt” also refers to a salt prepared from apilimod having a basic functional group, such as an amino functional group, and a pharmaceutically acceptable inorganic or organic acid.

The salts of the compounds described herein can be synthesized from the parent compound by conventional chemical methods such as methods described in Pharmaceutical Salts: Properties, Selection, and Use, P. Hemrich Stahl (Editor), Camille G. Wermuth (Editor), ISBN: 3-90639-026-8, August 2002. Generally, such salts can be prepared by reacting the parent compound (e.g., 2-[2-Pyridin-2-yl)-ethoxy]-4-N′-(3-methyl-benzilidene)-hydrazino]-6-(morpholin-4-yl)-pyrimidine) with the appropriate acid in water or in an organic solvent, or in a mixture of the two.

One salt form of a compound described herein can be converted to the free base and optionally to another salt form by methods well known to the skilled person. For example, the free base can be formed by passing the salt solution through a column containing an amine stationary phase (e.g. a Strata-NH₂ column). Alternatively, a solution of the salt in water can be treated with sodium bicarbonate to decompose the salt and precipitate out the free base. The free base may then be combined with another acid using routine methods.

As used herein, the term “polymorph” means solid crystalline forms of a compound of the present disclosure (e.g., 2-[2-Pyridin-2-yl)-ethoxy]-4-N′-(3-methyl-benzilidene)-hydrazino]-6-(morpholin-4-yl)-pyrimidine) or complex thereof. Different polymorphs of the same compound can exhibit different physical, chemical and/or spectroscopic properties. Different physical properties include, but are not limited to stability (e.g., to heat or light), compressibility and density (important in formulation and product manufacturing), and dissolution rates (which can affect bioavailability). Differences in stability can result from changes in chemical reactivity (e.g., differential oxidation, such that a dosage form discolors more rapidly when comprised of one polymorph than when comprised of another polymorph) or mechanical characteristics (e.g., tablets crumble on storage as a kinetically favored polymorph converts to thermodynamically more stable polymorph) or both (e.g., tablets of one polymorph are more susceptible to breakdown at high humidity). Different physical properties of polymorphs can affect their processing. For example, one polymorph might be more likely to form solvates or might be more difficult to filter or wash free of impurities than another due to, for example, the shape or size distribution of particles of it.

As used herein, the term “hydrate” means a compound of the present disclosure (e.g., 2-[2-Pyridin-2-yl)-ethoxy]-4-N′-(3-methyl-benzilidene)-hydrazino]-6-(morpholin-4-yl)-pyrimidine) or a salt thereof, which further includes a stoichiometric or non-stoichiometric amount of water bound by non-covalent intermolecular forces.

As used herein, the term “clathrate” means a compound of the present disclosure (e.g., 2-[2-Pyridin-2-yl)-ethoxy]-4-N′-(3-methyl-benzilidene)-hydrazino]-6-(morpholin-4-yl)-pyrimidine) or a salt thereof in the form of a crystal lattice that contains spaces (e.g., channels) that have a guest molecule (e.g., a solvent or water) trapped within.

As used herein, the term “prodrug” means a derivative of a compound described herein (e.g., 2-[2-Pyridin-2-yl)-ethoxy]-4-N′-(3-methyl-benzilidene)-hydrazino]-6-(morpholin-4-yl)-pyrimidine) that can hydrolyze, oxidize, or otherwise react under biological conditions (in vitro or in vivo) to provide a compound of the disclosure. Prodrugs may only become active upon such reaction under biological conditions, or they may have activity in their unreacted forms. Examples of prodrugs contemplated in this disclosure include, but are not limited to, analogs or derivatives of a compound described herein (e.g., 2-[2-Pyridin-2-yl)-ethoxy]-4-N′-(3-methyl-benzilidene)-hydrazine)-6-(morpholin-4-yl)-pyrimidine) that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of compounds of any one of the formulae disclosed herein that comprise —NO, —NO₂, —ONO, or —ONO₂ moieties. Prodrugs can typically be prepared using well-known methods, such as those described by Burger's Medicinal Chemistry and Drug Discovery (1995) 172-178, 949-982 (Manfred E. Wolff ed., 5th ed).

As used herein, the term “solvate” or “pharmaceutically acceptable solvate,” is a solvate formed from the association of one or more solvent molecules to one of the compounds disclosed herein (e.g., 2-[2-Pyridin-2-yl)-ethoxy]-4-N′-(3-methyl-benzilidene)-hydrazino]-6-(morpholin-4-yl)-pyrimidine). The term solvate includes hydrates (e.g., hemi-hydrate, mono-hydrate, dihydrate, trihydrate, tetrahydrate, and the like).

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound. As used herein, the term “derivative” refers to compounds that have a common core structure, and are substituted with various groups as described herein.

### Methods of Treatment and Diagnostic Methods

The present disclosure provides methods for the treatment of cancer in a subject having cancer cells overexpressing a microphthalmia (MiT) transcription factor. In embodiments, the methods comprise administering to the subject a therapeutically effective amount of apilimod, or a pharmaceutically acceptable salt, solvate, clathrate, hydrate, polymorph, prodrug, analog or derivative thereof. In embodiments, the cancer overexpresses an MiT transcription factor selected from TFEB, TFE3, TFEC, and MITF. In embodiments, the MiT transcription factor is selected from TFEB and TFE3, or both.

In embodiments, the present disclosure also provides diagnostic methods for identifying a cancer that is susceptible to apilimod treatment, the method comprising a step of assaying a sample of the cancer for the expression of one or more MiT transcription factors, where overexpression of one or more MiT transcription factors indicates that the cancer is susceptible to apilimod.

In embodiments, the cancer is brain cancer, glioma, sarcoma, breast cancer, lung cancer, non-small-cell lung cancer, mesothelioma, appendiceal cancer, genitourinary cancers, renal cell carcinoma, prostate cancer, bladder cancer, testicular cancer, penile cancer, cervical cancer, ovarian cancer, von Hippel Lindau disease, head and neck cancer, gastrointestinal cancer, hepatocellular carcinoma, gallbladder cancer, esophageal cancer, gastric cancer, colorectal cancer, pancreatic cancer, neuroendocrine tumors, thyroid tumor, pituitary tumor, adrenal tumor, hematological malignancy, or leukemia.

In embodiments, the cancer is a renal cancer, an alveolar soft part sarcoma or a perivascular epitheloid cell neoplasm having a TFE3 translocation.

In embodiments, the cancer is a renal cancer, a colorectal cancer, an endometrial cancer, or a gastric cancer having an FLCN inactivating mutation.

In one embodiment the renal cancer is a renal cell carcinoma (RCC). In one embodiment, the renal cell carcinoma is selected from the group consisting of clear cell renal cell carcinoma, papillary renal cell carcinoma, chromophobe renal cell carcinoma, other rare types of renal cell carcinoma (e.g., Collecting duct RCC, multilocular cystic RCC, medullary carcinoma, mucinous tubular and spindle cell carcinoma, neuroblastoma-associated RCC, unclassified renal cell carcinoma), and metastatic RCC. In one embodiment the renal cancer is selected from the group consisting of transitional cell carcinoma, Wilms tumor (nephroblastoma), renal sarcoma, and benign (non-cancerous) kidney tumors, renal adenoma, oncocytoma, and angiomyolipoma. In embodiments, the RCC is a subtype selected from papillary type I and type II, chromophobe, hybrid, oncocytoma, translocation, angiomyolipoma, oncocytic, medullary, and collecting duct carcinomas.

In one embodiment the cancer is a lymphoma. In one embodiment, the lymphoma is a B cell lymphoma. In one embodiment, the B cell lymphoma is selected from the group consisting of a Hodgkin's B cell lymphoma and a non-Hodgkin's B cell lymphoma. In one embodiment, the B cell lymphoma is a non-Hodgkin's B cell lymphoma selected from the group consisting of DLBCL, follicular lymphoma, marginal zone lymphoma (MZL) or mucosa associated lymphatic tissue lymphoma (MALT), small cell lymphocytic lymphoma (overlaps with chronic lymphocytic leukemia) and mantle cell lymphoma. In one embodiment, the B cell lymphoma is a non-Hodgkin's B cell lymphoma selected from the group consisting of Burkitt's lymphoma, Primary mediastinal (thymic) large B-cell lymphoma, Lymphoplasmacytic lymphoma, which may manifest as Waldenström macroglobulinemia, Nodal marginal zone B cell lymphoma (NMZL), Splenic marginal zone lymphoma (SMZL), Intravascular large B-cell lymphoma, Primary effusion lymphoma, Lymphomatoid granulomatosis, T cell/histiocyte-rich large B-cell lymphoma, Primary central nervous system lymphoma, Primary cutaneous diffuse large B-cell lymphoma, leg type (Primary cutaneous DLBCL, leg type), EBV positive diffuse large B-cell lymphoma of the elderly, Diffuse large B-cell lymphoma associated with inflammation, Intravascular large B-cell lymphoma, ALK-positive large B-cell lymphoma, and Plasmablastic lymphoma.

In one embodiment the cancer is a melanoma. Melanoma is a type of skin cancer which forms from melanocytes (pigment-containing cells in the skin), which are found in the epidermis of the skin. The epidermis is the upper or outer layer of the two main layers of cells that make up the skin and is separated from the deeper layers of the skin by the basement membrane. When a skin cancer such as melanoma becomes more advanced, it generally penetrated the epidermis and grows through the membrane into the deeper layers of the skin to gain access to the blood supply, which enables the tumor to metastasize.

There are four basic types of melanoma. Three of them begin in situ—meaning they occupy only the top layers of the skin, and sometimes become invasive; the fourth is invasive from the start. Invasive melanomas are more serious, as they have penetrated deeper into the skin and may have spread to other areas of the body.

Superficial spreading melanoma is by far the most common type, accounting for about 70 percent of all cases. This is the one most often seen in young people. As the name suggests, this melanoma grows along the top layer of the skin for a fairly long time before penetrating more deeply.

Lentigo maligna is similar to the superficial spreading type, as it also remains close to the skin surface for quite a while, and usually appears as a flat or mildly elevated mottled tan, brown or dark brown discoloration. This type of in situ melanoma is found most often in the elderly, arising on chronically sun-exposed, damaged skin on the face, ears, arms and upper trunk. When this cancer becomes invasive, it is referred to as lentigo maligna melanoma.

Acral lentiginous melanoma also spreads superficially before penetrating more deeply. It is quite different from the others, though, as it usually appears as a black or brown discoloration under the nails or on the soles of the feet or palms of the hands. This type of melanoma is sometimes found on dark-skinned people, and can often advance more quickly than superficial spreading melanoma and lentigo maligna. It is the most common melanoma in African-Americans and Asians, and the least common among Caucasians.

Nodular melanoma is usually invasive at the time it is first diagnosed. The malignancy is recognized when it becomes a bump. It is usually black, but occasionally is blue, gray, white, brown, tan, red or skin tone.

In one embodiment the cancer is a colorectal cancer. Colorectal cancer (also known as colon cancer, rectal cancer, or bowel cancer) is the development of cancer in the colon or rectum. Colon cancer is staged according to the TNM staging system. The TNM system is one of the most widely used cancer staging systems and has been adopted by the Union for International Cancer Control (UICC) and the American Joint Committee on Cancer (AJCC). The TNM system is based on the size and/or extent (reach) of the primary tumor (T), the amount of spread to nearby lymph nodes (N), and the presence of metastasis (M) or secondary tumors formed by the spread of cancer cells to other parts of the body. A number is added to each letter to indicate the size and/or extent of the primary tumor and the degree of cancer spread.

The present disclosure also provides methods comprising combination therapy for the treatment of cancer. As used herein, “combination therapy” or “co-therapy” includes the administration of a therapeutically effective amount of apilimod as part of a specific treatment regimen intended to provide the beneficial effect from the co-action of the apilimod and the additional active agent. The at least one additional agent may be a therapeutic agent or a non-therapeutic agent. The beneficial effect of the combination includes, but is not limited to, pharmacokinetic or pharmacodynamic co-action resulting from the combination of therapeutic compounds. The beneficial effect of the combination may also relate to the mitigation of a toxicity, side effect, or adverse event associated with another agent in the combination. “Combination therapy” is not intended to encompass the administration of two or more of these therapeutic compounds as part of separate monotherapy regimens that incidentally and arbitrarily result in a beneficial effect that was not intended or predicted.

The at least one additional active agent may be a therapeutic agent, for example an anti-cancer agent or a cancer chemotherapeutic agent, or a non-therapeutic agent, and combinations thereof. With respect to therapeutic agents, the beneficial effect of the combination includes, but is not limited to, pharmacokinetic or pharmacodynamic co-action resulting from the combination of therapeutically active compounds. With respect to nontherapeutic agents, the beneficial effect of the combination may relate to the mitigation of a toxicity, side effect, or adverse event associated with a therapeutically active agent in the combination.

In one embodiment, the at least one additional agent is a non-therapeutic agent which mitigates one or more side effects of an apilimod composition, the one or more side effects selected from any of nausea, vomiting, headache, dizziness, lightheadedness, drowsiness and stress. In one aspect of this embodiment, the non-therapeutic agent is an antagonist of a serotonin receptor, also known as 5-hydroxytryptamine receptors or 5-HT receptors. In one aspect, the non-therapeutic agent is an antagonist of a 5-HT3 or 5-HT1a receptor. In one aspect, the non-therapeutic agent is selected from the group consisting of ondansetron, granisetron, dolasetron and palonosetron. In another aspect, the non-therapeutic agent is selected from the group consisting of pindolol and risperidone.

In embodiments, the at least one additional agent is a therapeutic agent. In one embodiment, the therapeutic agent is an anti-cancer agent as described in more detail below.

In the context of combination therapy, administration of apilimod, or a pharmaceutically acceptable salt, solvate, clathrate, hydrate, polymorph, metabolite, prodrug, analog or derivative thereof, may be simultaneous with or sequential to the administration of the one or more additional active agents. In another embodiment, administration of the different components of a combination therapy may be at different frequencies. The one or more additional agents may be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a compound of the present disclosure.

The one or more additional active agents can be formulated for co-administration with apilimod in a single dosage form, as described in greater detail herein. The one or more additional active agents can be administered separately from the dosage form that comprises the apilimod. When the additional active agent is administered separately from the apilimod, it can be by the same or a different route of administration as the apilimod.

Preferably, the administration of a composition comprising apilimod in combination with one or more additional active agents provides a synergistic response in the subject being treated. In this context, the term “synergistic” refers to the efficacy of the combination being more effective than the additive effects of either single therapy alone. The synergistic effect of a combination therapy according to the disclosure can permit the use of lower dosages and/or less frequent administration of at least one agent in the combination compared to its dose and/or frequency outside of the combination. Additional beneficial effects of the combination can be manifested in the avoidance or reduction of adverse or unwanted side effects associated with the use of either therapy in the combination alone (also referred to as monotherapy).

“Combination therapy” also embraces the administration of the compounds of the present disclosure in further combination with non-drug therapies (e.g., surgery or radiation treatment). Where the combination therapy further comprises a non-drug treatment, the non-drug treatment may be conducted at any suitable time so long as a beneficial effect from the co-action of the combination of the therapeutic compounds and non-drug treatment is achieved. For example, in appropriate cases, the beneficial effect is still achieved when the non-drug treatment is temporally removed from the administration of the therapeutic compounds, perhaps by days or even weeks.

The non-drug treatment can be selected from chemotherapy, radiation therapy, hormonal therapy, anti-estrogen therapy, gene therapy, surgery (e.g. radical nephrectomy, partial nephrectomy, laparoscopic and robotic surgery), radiofrequency ablation, and cryoablation. For example, a non-drug therapy is the removal of an ovary (e.g., to reduce the level of estrogen in the body), thoracentesis (e.g., to remove fluid from the chest), paracentesis (e.g., to remove fluid from the abdomen), surgery to remove or shrink angiomyolipomas, lung transplantation (and optionally with an antibiotic to prevent infection due to transplantation), or oxygen therapy (e.g., through a nasal cannula containing two small plastic tubes or prongs that are placed in both nostrils, through a face mask that fits over the nose and mouth, or through a small tube inserted into the windpipe through the front of the neck, also called transtracheal oxygen therapy).

In one embodiment, the at least one additional agent is an agent which mitigates one or more side effects of apilimod selected from any of nausea, vomiting, headache, dizziness, lightheadedness, drowsiness and stress. In one aspect of this embodiment, the additional agent is an antagonist of a serotonin receptors, also known as 5-hydroxytryptamine receptors or 5-HT receptors. In one aspect, the additional agent is an antagonist of a 5-HT₃ or 5-HT₁ₐ receptor. In one aspect, the agent is selected from the group consisting of ondansetron, granisetron, dolasetron and palonosetron. In another aspect, the agent is selected from the group consisting of pindolol and risperidone.

In embodiments, the at least one additional agent is an anti-cancer agent. In embodiments where the cancer is a renal cancer, the anti-cancer agent may be selected from a VEGF inhibitor such as sunitinib, pazopanib, bevacizumab, sorafenib, cabozantinb and axitinib or an mTOR inhibitor such as everolimus or temsirolimus.

In one embodiment, the anti-cancer agent is selected from taxol, vincristine, doxorubicin, temsirolimus, carboplatin, ofatumumab, rituximab, and combinations thereof.

In one embodiment, the at least one additional agent is selected from chlorambucil, ifosphamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibrilumomab, lositumomab, borlezomib, pentostatin, endostatin, or a combination thereof.

In one embodiment, the at least one additional agent is selected from Afinitor (Everolimus), Aldesleukin, Avastin (Bevacizumab), Axitinib, Bevacizumab, Everolimus, IL-2 (Aldesleukin), Inlyta (Axitinib), Interleukin-2 (Aldesleukin), Nexavar (Sorafenib Tosylate), Pazopanib, Hydrochloride, Proleukin (Aldesleukin), Sorafenib Tosylate, Sunitinib Malate, Sutent (Sunitinib Malate), Temsirolimus, Torisel (Temsirolimus), Votrient (Pazopanib Hydrochloride), or combination thereof.

In one embodiment, the at least one additional agent is directed towards targeted therapy, wherein the treatment targets the cancer's specific genes, proteins, or the tissue environment that contributes to cancer growth and survival. This type of treatment blocks the growth and spread of cancer cells while limiting damage to healthy cells.

In one embodiment, the at least one additional agent is directed towards anti-angiogenesis therapy, wherein the treatment focuses on stopping angiogenesis, which is the process of making new blood vessels. Because a tumor needs the nutrients delivered by blood vessels to grow and spread, the goal of anti-angiogenesis therapies is to “starve” the tumor. One anti-angiogenic drug, bevacizumab (Avastin), has been shown to slow tumor growth for people with metastatic renal carcinoma. Bevacizumab combined with interferon slows tumor growth and spread.

In one embodiment, the at least one additional agent is directed towards immunotherapy, also called biologic therapy, is designed to boost the body's natural defenses to fight cancer. It uses materials made either by the body or in a laboratory to improve, target, or restore immune system function. For example, Interleukin-2 (IL-2) is a drug that has been used to treat kidney cancer as well as AM0010, and interleukin-15. They are cellular hormones called cytokines produced by white blood cells and are important in immune system function, including the destruction of tumor cells. Alpha-interferon is another type of immunotherapy used to treat kidney cancer that has spread. Interferon appears to change the proteins on the surface of cancer cells and slow their growth. Many combination therapies of IL-2 and alpha-interferon for patients with advanced kidney cancer combined with chemotherapy are more effective than IL-2 or interferon alone.

In embodiments, the at least one additional agent is a PD-1/PDL-1 pathway inhibitor. In embodiments, the PD-1/PDL-1 pathway inhibitor is selected from pembrolizumab (Keytruda, MK-3475), avelumab, atezolizumab (MPDL3280A), nivolumab (BMS-936558), pidilizumab (CT-011), MSB0010718C, and MEDI4736.

In embodiments, the at least one additional agent is a check point inhibitor. Treatment with these compounds work by targeting molecules that serve as checks and balances on immune responses. By blocking these inhibitory molecules or, alternatively, activating stimulatory molecules, these treatments are designed to unleash or enhance pre-existing anti-cancer immune responses. In embodiments, the check point inhibitor may be selected from an antibody such as PD-1, anti-CD27, B7-H3, KIR, LAG-3, 4-1BB/CD137, GITR, pembrolizumab (Keytruda, a PD-1 antibody), MPDL3280A (a PD-L1 antibody), varlilumab (CDX-1127, an anti-CD27 antibody), MGA217 (an antibody that targets B7-H3), lirilumab (a KIR antibody), BMS-986016 (a LAG-3 antibody), urelumab (a 4-1BB/CD137 antibody), anti-TIM3 (a TIM3 antibody), MEDI-0562 (a OX40 antibody), SEA-CD40 (a CD40 antibody), TRX518 (a GITR antibody), and MK-4166 (a GITR antibody).

In embodiments, the at least one additional agent is a cancer vaccine, designed to elicit an immune response against tumor-specific or tumor-associated antigens, encouraging the immune system to attack cancer cells bearing these antigens. In embodiments, the cancer vaccine may be selected from AGS-003, DCVax, and NY-ESO-1.

In embodiments, the at least one additional agent is an immunostimulant, such as a recombinant protein, used to activate the immune system to attack cancer cells. In embodiments, the immunostimulant is denenicokin (recombinant IL-21).

In embodiments, the at least one additional agent is a small molecule that modulates the immune system to encourage elimination of cancer cells. In embodiments, the small molecule may be selected from epacadostat (an IDO inhibitor) and PLX3397 (an inhibitor of CSF-1R).

In embodiments, the at least one additional agent may be the patient's own immune cells which have been removed from a patient, genetically modified or treated with chemicals to enhance their activity, and then re-introduced into the patient with the goal of improving the immune system's anti-cancer response.

In the context of the methods described herein, the amount of apilimod administered to the subject is a therapeutically effective amount. The term “therapeutically effective amount” refers to an amount sufficient to treat, ameliorate a symptom of, reduce the severity of, or reduce the duration of the disease or disorder being treated or enhance or improve the therapeutic effect of another therapy, or sufficient to exhibit a detectable therapeutic effect in the subject. In one embodiment, the therapeutically effective amount of an apilimod composition is the amount effective to inhibit PIKfyve kinase activity.

An effective amount of apilimod can range from about 0.001 mg/kg to about 1000 mg/kg, about 0.01 mg/kg to about 100 mg·kg, about 10 mg/kg to about 250 mg/kg, about 0.1 mg/kg to about 15 mg/kg; or any range in which the low end of the range is any amount between 0.001 mg/kg and 900 mg/kg and the upper end of the range is any amount between 0.1 mg/kg and 1000 mg/kg (e.g., 0.005 mg/kg and 200 mg/kg, 0.5 mg/kg and 20 mg/kg). Effective doses will also vary, as recognized by those skilled in the art, depending on the diseases treated, route of administration, excipient usage, and the possibility of co-usage with other therapeutic treatments such as use of other agents. See, e.g., U.S. Pat. No. 7,863,270, incorporated herein by reference.

In more specific aspects, the apilimod is administered at a dosage regimen of 30-1000 mg/day (e.g., 30, 35, 40, 45, 50, 55, 60, 65, 70, 75, 80, 85, 90, 95, 100, 125, 150, 175, 200, 225, 250, 275, or 300 mg/day) for at least 1 week (e.g., 1, 2, 3, 4, 5, 6, 7, 8, 9, 10, 11, 12, 36, 48, or more weeks). Preferably, apilimod is administered at a dosage regimen of 100-1000 mg/day for 4 or 16 weeks. Alternatively or subsequently, apilimod is administered at a dosage regimen of 100-300 mg twice a day for 8 weeks, or optionally, for 52 weeks. Alternatively or subsequently, an apilimod composition is administered at a dosage regimen of 50 mg-1000 mg twice a day for 8 weeks, or optionally, for 52 weeks.

An effective amount of the apilimod composition can be administered once daily, from two to five times daily, up to two times or up to three times daily, or up to eight times daily. In one embodiment, the apilimod composition is administered thrice daily, twice daily, once daily, fourteen days on (four times daily, thrice daily or twice daily, or once daily) and 7 days off in a 3-week cycle, up to five or seven days on (four times daily, thrice daily or twice daily, or once daily) and 14-16 days off in 3 week cycle, or once every two days, or once a week, or once every 2 weeks, or once every 3 weeks.

In accordance with the methods described herein, a “subject in need thereof” is a subject having renal cancer, or a subject having an increased risk of developing renal cancer relative to the population at large. The subject in need thereof can be one that is “non-responsive” or “refractory” to a currently available therapy for the cancer. In this context, the terms “non-responsive” and “refractory” refer to the subject's response to therapy as not clinically adequate to relieve one or more symptoms associated with the disease or disorder. In one aspect of the methods described here, the subject in need thereof is a subject having cancer whose cancer is refractory to standard therapy or whose cancer has recurred following standard treatment.

A “subject” includes a mammal. The mammal can be e.g., any mammal, e.g., a human, primate, vertebrate, bird, mouse, rat, fowl, dog, cat, cow, horse, goat, camel, sheep or a pig. Preferably, the mammal is a human. The term “patient” refers to a human subject.

The present disclosure also provides a monotherapy for the treatment of renal cancer as described herein. As used herein, “monotherapy” refers to the administration of a single active or therapeutic compound to a subject in need thereof.

As used herein, “treatment”, “treating” or “treat” describes the management and care of a patient for the purpose of combating a disease, condition, or disorder and includes the administration of apilimod to alleviate the symptoms or complications of a disease, condition or disorder, or to eliminate the disease, condition or disorder.

As used herein, “prevention”, “preventing” or “prevent” describes reducing or eliminating the onset of the symptoms or complications of the disease, condition or disorder and includes the administration of apilimod to reduce the onset, development or recurrence of symptoms of the disease, condition or disorder.

In one embodiment, the administration of apilimod leads to the elimination of a symptom or complication of the cancer being treated, however elimination of the cancer is not required. In one embodiment, the severity of the symptom is decreased. In the context of cancer, such symptoms may include clinical markers of severity or progression including the degree to which a tumor secretes growth factors, degrades the extracellular matrix, becomes vascularized, loses adhesion to juxtaposed tissues, or metastasizes, as well as the number of metastases.

Treating cancer according to the methods described herein can result in a reduction in size of a tumor. A reduction in size of a tumor may also be referred to as “tumor regression”. Preferably, after treatment, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. The size of a tumor may be measured as a diameter of the tumor.

Treating cancer according to the methods described herein can result in a reduction in tumor volume. Preferably, after treatment, tumor volume is reduced by 5% or greater relative to its size prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

Treating cancer according to the methods described herein can result in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

Treating cancer according to the methods described herein can result in a decrease in number of metastatic lesions in other tissues or organs distant from the primary tumor site. Preferably, after treatment, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

Treating cancer according to the methods described herein can result in an increase in average survival time of a population of treated subjects in comparison to a population receiving carrier alone. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer according to the methods described herein can result in an increase in average survival lime of a population of treated subjects in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer according to the methods described herein can result in increase in average survival time of a population of treated subjects in comparison to a population receiving monotherapy with a drug that is not apilimod. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer according to the methods described herein can result in a decrease in the mortality rate of a population of treated subjects in comparison to a population receiving carrier alone. Treating a disorder, disease or condition according to the methods described herein can result in a decrease in the mortality rate of a population of treated subjects in comparison to an untreated population. Treating a disorder, disease or condition according to the methods described herein can result in a decrease in the mortality rate of a population of treated subjects in comparison to a population receiving monotherapy with a drug that is not apilimod. Preferably, the mortality rate is decreased by more than 2%; more preferably, by more than 5%; more preferably, by more than 10%; and most preferably, by more than 25%. A decrease in the mortality rate of a population of treated subjects may be measured by any reproducible means. A decrease in the mortality rate of a population may be measured, for example, by calculating for a population the average number of disease-related deaths per unit lime following initiation of treatment with an active compound. A decrease in the mortality rate of a population may also be measured, for example, by calculating for a population the average number of disease-related deaths per unit time following completion of a first round of treatment with an active compound.

Treating cancer according to the methods described herein can result in a decrease in tumor growth rate. Preferably, after treatment, tumor growth rate is reduced by at least 5% relative to number prior to treatment; more preferably, tumor growth rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. Tumor growth rate can be measured according to a change in tumor diameter per unit time. In one embodiment, after treatment the tumor growth rate may be about zero and is determined to maintain the same size, e.g., the tumor has stopped growing.

Treating cancer according to the methods described herein can result in a decrease in tumor regrowth. Preferably, after treatment, tumor regrowth is less than 5%; more preferably, tumor regrowth is less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by failure of tumors to reoccur after treatment has stopped.

Treating or preventing a cell proliferative disorder according to the methods described herein can result in a reduction in the rate of cellular proliferation. Preferably, after treatment, the rate of cellular proliferation is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The rate of cellular proliferation may be measured by any reproducible means of measurement. The rate of cellular proliferation is measured, for example, by measuring the number of dividing cells in a tissue sample per unit time.

Treating or preventing a cell proliferative disorder according to the methods described herein can result in a reduction in the proportion of proliferating cells. Preferably, after treatment, the proportion of proliferating cells is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The proportion of proliferating cells may be measured by any reproducible means of measurement. Preferably, the proportion of proliferating cells is measured, for example, by quantifying the number of dividing cells relative to the number of nondividing cells in a tissue sample. The proportion of proliferating cells can be equivalent to the mitotic index.

Treating or preventing a cell proliferative disorder according to the methods described herein can result in a decrease in the size of an area or zone of cellular proliferation. Preferably, after treatment, size of an area or zone of cellular proliferation is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. The size of an area or zone of cellular proliferation may be measured by any reproducible means of measurement. The size of an area or zone of cellular proliferation may be measured as a diameter or width of an area or zone of cellular proliferation.

Treating or preventing a cell proliferative disorder according to the methods described herein can result in a decrease in the number or proportion of cells having an abnormal appearance or morphology. Preferably, after treatment, the number of cells having an abnormal morphology is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. An abnormal cellular appearance or morphology may be measured by any reproducible means of measurement. An abnormal cellular morphology can be measured by microscopy, e.g., using an inverted tissue culture microscope. An abnormal cellular morphology can take the form of nuclear pleiomorphism.

As used herein, the term “selectively” means tending to occur at a higher frequency in one population than in another population. The compared populations can be cell populations. Preferably, apilimod acts selectively on a hyper-proliferating cells or abnormally proliferating cells, compared to normal cells. As used herein, a “normal cell” is a cell that cannot be classified as part of a “cell proliferative disorder”. A normal cell lacks unregulated or abnormal growth, or both, that can lead to the development of an unwanted condition or disease. Preferably, a normal cell possesses normally functioning cell cycle checkpoint control mechanisms. Preferably, apilimod, acts selectively to modulate one molecular target (e.g., a target kinase) but does not significantly modulate another molecular target (e.g., a non-target kinase). The disclosure also provides a method for selectively inhibiting the activity of an enzyme, such as a kinase. Preferably, an event occurs selectively in population A relative to population B if it occurs greater than two times more frequently in population A as compared to population B. An event occurs selectively if it occurs greater than five times more frequently in population A. An event occurs selectively if it occurs greater than ten times more frequently in population A; more preferably, greater than fifty times; even more preferably, greater than 100 times; and most preferably, greater than 1000 times more frequently in population A as compared to population B. For example, cell death would be said to occur selectively in diseased or hyper-proliferating cells if it occurred greater than twice as frequently in diseased or hyper-proliferating cells as compared to normal cells.

### Pharmaceutical Compositions and Formulations

The present disclosure provides pharmaceutical compositions comprising an amount of apilimod, or a pharmaceutically acceptable salt, solvate, clathrate, hydrate, polymorph, metabolite, prodrug, analog or derivative thereof, in combination with at least one pharmaceutically acceptable excipient or carrier, wherein the amount is effective for the treatment of a cancer as described herein, and/or effective to inhibit PIKfyve in the cancer cells of a subject having cancer.

In one embodiment, the apilimod is apilimod free base. In one embodiment, the apilimod is apilimod dimesylate.

In one embodiment, the apilimod is combined with at least one additional active agent in a single dosage form. In one embodiment, the composition further comprises an antioxidant.

In embodiments, the at least one additional active agent is selected from the group consisting of an alkylating agent, an intercalating agent, a tubulin binding agent, a corticosteroid, and combinations thereof. In one embodiment, the at least one additional active agent is a therapeutic agent selected from the group consisting of ibrutinib, rituximab, doxorubicin, prednisolone, vincristine, velcade, and everolimus, and combinations thereof. In one embodiment, the at least one additional active agent is a therapeutic agent selected from cyclophosphamide, hydroxydaunorubicin (also referred to as doxorubicin or Adriamycin™), vincristine (also referred to as Oncovin™), prednisone, prednisolone, and combinations thereof.

In embodiments, the at least one additional active agent is a non-therapeutic agent selected to ameliorate one or more side effects of the apilimod composition. In one embodiment, the nontherapeutic agent is selected from the group consisting of ondansetron, granisetron, dolasetron and palonosetron. In one embodiment, the non-therapeutic agent is selected from the group consisting of pindolol and risperidone.

In embodiments, at least one additional agent is a PD-1/PDL-1 pathway inhibitor. In embodiments, the PD-1/PDL-1 pathway inhibitor is selected from pembrolizumab (Keytruda), avelumab, atezolizumab (MPDL3280A), nivolumab (BMS-936558), pidilizumab (CT-011), MSB0010718C, and MEDI4736.

In embodiments, the at least one additional active agent is selected from an inhibitor of the mTOR pathway, a TKI inhibitor, a PI3K inhibitor, a dual PI3K/mTOR inhibitor, a SRC inhibitor, a VEGF inhibitor, a Janus kinase (JAK) inhibitor, a Raf inhibitor, an Erk inhibitor, a farnesyltransferase inhibitor, a c-MET inhibitor, a histone deacelylase inhibitor, an anti-mitotic agent, a multi-drug resistance efflux inhibitor, an antibiotic, and a cytokine. In one embodiment, the second therapeutic agent is a therapeutic cytokine. In one embodiment, the second therapeutic agent is Interleukin-2. In another embodiment, the second therapeutic agent is selected from a tyrosine kinase inhibitor (e.g., everolimus, bevacizumab).

In embodiments, the mTOR inhibitor is selected from the group consisting of rapamycin (also referred to as sirolimus), everolimus, temsirolimus, ridaforolimus, umirolimus, zotarolimus, AZD8055, INK128, WYE-132, Torin-1, pyrazolopyrimidine analogs PP242, PP30, PP487, PP121, KU0063794, KU-BMCL-200908069-1, Wyeth-BMCL-200910075-9b, INK-128, XL388, AZD8055, P2281, and P529. See, e.g., Liu et al. Drug Disc. Today Ther. Strateg., 6(2): 47-55 (2009).

In embodiments, the mTOR inhibitor is trans-4-[4-amino-5-(7-methoxy-1H-indol-2-yl)imidazo[5,1-f][1,2,4]triazin-7-yl]cyclohexane carboxylic acid (also known as OSI-027), and any salts, solvates, hydrates, and other physical forms, crystalline or amorphous, thereof. See US 2007/0112005. OSI-027 can be prepared according to US 2007/0112005, incorporated herein by reference. In one embodiment, the mTOR inhibitor is OXA-01. See e.g., WO 2013152342 A1.

In embodiments, the PI3K inhibitor is selected from the group consisting of GS-1101 (Idelalisib), GDC0941 (Pictilisib), LY294002, BKM120 (Buparlisib), PI-103, TGX-221, IC-87114, XL 147, ZSTK474, BYL719, AS-605240, PIK-75, 3-methyladenine, A66, PIK-93, PIK-90, AZD6482, IPI-145 (Duvelisib), TG100-115, AS-252424, PIK294, AS-604850, GSK2636771, BAY 80-6946 (Copanlisib), CH5132799, CAY10505, PIK-293, TG100713, CZC24832 and HS-173.

In embodiments, the dual PI3K/mTOR inhibitor is selected from the group consisting of, GDC-094, WAY-001, WYE-354, WAY-600, WYE-687, Wyeth-BMCL-200910075-16b, Wyeth-BMCL-200910096-27, KU0063794 and KUBMCL-200908069-5, NVP-BEZ235, XL-765, PF-04691502, GDC-0980 (Apitolisib), GSK1059615, PF-05212384, BGT226, PKI-402, VS-558 and GSK2126458. See, e.g., Liu et al. Drug Disc. Today Ther. Strateg., 6(2): 47-55 (2009), incorporated herein by reference.

In embodiments, the mTOR pathway inhibitor is a polypeptide (e.g., an antibody or fragment thereof) or a nucleic acid (e.g., a double-stranded small interfering RNA, a short hairpin RNA, a micro-RNA, an antisense oligonucleotide, a locked nucleic acid, or an aptamer) that binds to and inhibits the expression level or activity or a protein (or nucleic acid encoding the protein) in the mTOR pathway. For example, the polypeptide or nucleic acid inhibits mTOR Complex 1 (mTORC1), regulatory-associated protein of mTOR (Raptor), mammalian lethal with SEC13 protein 8 (MLST8), proline-rich Akt substrate of 40 kDa (PRAS40), DEP domain-containing mTOR-interacting protein (DEPTOR), mTOR Complex 2 (mTORC2), rapamycin-insensitive companion of mTOR (RICTOR), G protein beta subunit-like (GβL), mammalian stress-activated protein kinase interacting protein 1 (mSIN1), paxillin, RhoA, Ras-related C3 botulinum toxin substrate 1 (Rac1), Cell division control protein 42 homolog (Cdc42), protein kinase C α (PKCα), the serine/threonine protein kinase Akt, phosphoinositide 3-kinase (PI3K), p70S6K, Ras, and/or eukaryotic translation initiation factor 4E (eIF4E)-binding proteins (4EBPs), or the nucleic acid encoding one of these proteins.

In embodiments, the SRC inhibitor is selected from the group consisting of bosutinib, saracatinib, dasatinib, ponatinib, KX2-391, XL-228, TG100435/TG100855, and DCC2036. See, e.g., Puls et al. Oncologist. 2011 May; 16(5): 566-578. In one embodiment, the SRC inhibitor is a polypeptide (e.g., an antibody or fragment thereof) or nucleic acid (e.g., a double-stranded small interfering RNA, a short hairpin RNA, a micro-RNA, an antisense oligonucleotide, a locked nucleic acid, or an aptamer) that binds to and inhibits the expression level or activity of the SRC protein or a nucleic acid encoding the SRC protein.

In embodiments, the VEGF inhibitor is selected from bevacizumab, sunitinib, pazopanib, axitinib, sorafenib, regorafenib, lenvatinib, and motesanib. In one embodiment, the VEGF inhibitor is a polypeptide (e.g., an antibody or fragment thereof) or nucleic acid (e.g., a double-stranded small interfering RNA, a short hairpin RNA, a micro-RNA, an antisense oligonucleotide, a morpholino, a locked nucleic acid, or an aptamer) that binds to and inhibits the expression level or activity of a VEGF protein, a VEGF receptor protein, or a nucleic acid encoding one of these proteins. For example, the VEGF inhibitor is a soluble VEGF receptor (e.g., a soluble VEGF-C/D receptor (sVEGFR-3)).

In embodiments, the JAK inhibitor is selected from facitinib, ruxolitinib, baricitinib, CYT387 (CAS number 1056634-68-4), lestaurtinib, pacritinib, and TG101348 (CAS number 936091-26-8). In one embodiment, the JAK inhibitor is a polypeptide (e.g., an antibody or fragment thereof) or nucleic acid (e.g., a double-stranded small interfering RNA, a short hairpin RNA, a micro-RNA, an antisense oligonucleotide, a morpholino, a locked nucleic acid, or an aptamer) that binds to and inhibits the expression level or activity of a JAK (e.g., JAK1, JAK2, JAK3, or TYK2) or a nucleic acid encoding the JAK protein.

In embodiments, the Raf inhibitor is selected from PLX4032 (vemurafenib), sorafenib, PLX-4720, GSK2118436 (dabrafenib), GDC-0879, RAF265, AZ 628, NVP-BHG712, SB90885, ZM 336372, GW5074, TAK-632, CEP-32496 and LGX818 (Encorafenib). In one embodiment, the Raf inhibitor is a polypeptide (e.g., an antibody or fragment thereof) or nucleic acid (e.g., a double-stranded small interfering RNA, a short hairpin RNA, a micro-RNA, an antisense oligonucleotide, a morpholino, a locked nucleic acid, or an aptamer) that binds to and inhibits the expression level or activity of a Raf (e.g., A-Raf, B-Raf, C-Raf) or a nucleic acid encoding the Raf protein. In one embodiment, the MEK inhibitor is selected from AZD6244 (Sclumetinib), PD0325901, GSK1120212 (Trametinib), U0126-EtOH, PD184352, RDEA119 (Rafametinib), PD98059, BIX 02189, MEK162 (Binimetinib), AS-703026 (Pimasertib), SL-327, BIX02188, AZD8330, TAK-733 and PD318088. In one embodiment, the MEK inhibitor is a polypeptide (e.g., an antibody or fragment thereof) or nucleic acid (e.g., a double-stranded small interfering RNA, a short hairpin RNA, a micro-RNA, an anti sense oligonucleotide, a morpholino, a locked nucleic acid, or an aptamer) that binds to and inhibits the expression level or activity of a MEK (e.g., MEK-1, MEK-2) or a nucleic acid encoding the MEK protein.

In embodiments, the Akt inhibitor is selected from MK-2206, KRX-0401 (perifosine), GSK690693, GDC-0068 (Ipatasertib), AZD5363, CCT128930, A-674563, PHT-427. In one embodiment, the Akt inhibitor is a polypeptide (e.g., an antibody or fragment thereof) or nucleic acid (e.g., a double-stranded small interfering RNA, a short hairpin RNA, a micro-RNA, an antisense oligonucleotide, a morpholino, a locked nucleic acid, or an aptamer) that binds to and inhibits the expression level or activity of a Akt (e.g., Akt-1. Akt-2, Akt-3) or a nucleic acid encoding the Akt protein.

In embodiments, the farnesyltransferase inhibitor is selected from LB42708 or tipifarnib. In one embodiment, the farnesyltransferase inhibitor is a polypeptide (e.g., an antibody or fragment thereof) or nucleic acid (e.g., a double-stranded small interfering RNA, a short hairpin RNA, a micro-RNA, an antisense oligonucleotide, a morpholino, a locked nucleic acid, or an aptamer) that binds to and inhibits the expression level or activity of farnesyltransferase or a nucleic acid encoding the farnesyltransferase protein.

In one embodiment, the c-MET inhibitor is selected from crizotinib, tivantinib, cabozantinib, foretinib. In one embodiment, the c-MET inhibitor is a polypeptide (e.g., an antibody or fragment thereof, exemplified by onartuzumab) or nucleic acid (e.g., a double-stranded small interfering RNA, a short hairpin RNA, a micro-RNA, an antisense oligonucleotide, a morpholino, a locked nucleic acid, or an aptamer) that binds to and inhibits the expression level or activity of c-MET or a nucleic acid encoding the c-MET protein or the HGF ligand, such as ficlatuzumab or rilotumumab.

In one embodiment, the histone modulating inhibitor is selected from anacardic acid, C646, MG149 (histone acetyltransferase), GSK J4 Hcl (histone demethylase), GSK343 (active against EZH2), BIX 01294 (histone methyltransferase), MK0683 (Vorinostat), MS275 (Entinostat), LBH589 (Panobinostat), Trichostatin A, MGCD0103 (Mocetinostat), Tasquinimod, TMP269, Nexturastat A, RG2833, PDX101 (Belinostat).

In embodiments, the anti-mitotic agent is selected from Griseofulvin, vinorelbine tartrate, paclitaxel, docetaxel, vincristine, vinblastine, Epothilonc A. Epothilonc B, ABT-751, CYT997 (Lexibulin), vinflunine tartrate, Fosbrctabulin, GSK461364, ON-01910 (Rigosertib), Ro3280, BI2536, NMS-P937, BI 6727 (Volasertib), HMN-214 and MLN0905.

In embodiments, the tyrosine kinase inhibitor is selected from Votrient, Axitinib, Bortezomib, Bosutinib, Carfilzomib, Crizotinib, Dabrafenib, Dasatinib, Erlotinib, Gefitinib, Ibrutinib, Imatinib, Lapatinib, Nilotinib, Pegaptanib, Ponatinib, Regorafenib, Ruxolitinib, Sorafenib, Sunitinib, Trametinib, Vandetanib, Vemurafenib, and Vismodegib.

In one embodiment, the polyether antibiotic is selected from sodium monensin, nigericin, valinomycin, salinomycin.

A “pharmaceutical composition” is a formulation containing the compounds described herein in a pharmaceutically acceptable form suitable for administration to a subject. As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. Examples of pharmaceutically acceptable excipients include, without limitation, sterile liquids, water, buffered saline, ethanol, polyol (for example, glycerol, propylene glycol, liquid polyethylene glycol and the like), oils, detergents, suspending agents, carbohydrates (e.g., glucose, lactose, sucrose or dextran), antioxidants (e.g., ascorbic acid or glutathione), chelating agents, low molecular weight proteins, or suitable mixtures thereof.

A pharmaceutical composition can be provided in bulk or in dosage unit form. It is especially advantageous to formulate pharmaceutical compositions in dosage unit form for ease of administration and uniformity of dosage. The term “dosage unit form” as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved. A dosage unit form can be an ampoule, a vial, a suppository, a dragee, a tablet, a capsule, an IV bag, or a single pump on an aerosol inhaler.

In therapeutic applications, the dosages vary depending on the agent, the age, weight, and clinical condition of the recipient patient, and the experience and judgment of the clinician or practitioner administering the therapy, among other factors affecting the selected dosage. Generally, the dose should be a therapeutically effective amount. Dosages can be provided in mg/kg/day units of measurement (which dose may be adjusted for the patient's weight in kg, body surface area in m², and age in years). An effective amount of a pharmaceutical composition is that which provides an objectively identifiable improvement as noted by the clinician or other qualified observer. For example, alleviating a symptom of a disorder, disease or condition. As used herein, the term “dosage effective manner” refers to amount of a pharmaceutical composition to produce the desired biological effect in a subject or cell.

For example, the dosage unit form can comprise 1 nanogram to 2 milligrams, or 0.1 milligrams to 2 grams; or from 10 milligrams to 1 gram, or from 50 milligrams to 500 milligrams or from 1 microgram to 20 milligrams; or from 1 microgram to 10 milligrams; or from 0.1 milligrams to 2 milligrams.

The pharmaceutical compositions can take any suitable form (e.g, liquids, aerosols, solutions, inhalants, mists, sprays; or solids, powders, ointments, pastes, creams, lotions, gels, patches and the like) for administration by any desired route (e.g, pulmonary, inhalation, intranasal, oral, buccal, sublingual, parenteral, subcutaneous, intravenous, intramuscular, intraperitoneal, intrapleural, intrathecal, transdermal, transmucosal, rectal, and the like). For example, a pharmaceutical composition of the disclosure may be in the form of an aqueous solution or powder for aerosol administration by inhalation or insufflation (either through the mouth or the nose), in the form of a tablet or capsule for oral administration; in the form of a sterile aqueous solution or dispersion suitable for administration by either direct injection or by addition to sterile infusion fluids for intravenous infusion; or in the form of a lotion, cream, foam, patch, suspension, solution, or suppository for transdermal or transmucosal administration.

A pharmaceutical composition can be in the form of an orally acceptable dosage form including, but not limited to, capsules, tablets, buccal forms, troches, lozenges, and oral liquids in the form of emulsions, aqueous suspensions, dispersions or solutions. Capsules may contain mixtures of a compound of the present disclosure with inert fillers and/or diluents such as the pharmaceutically acceptable starches (e.g., corn, potato or tapioca starch), sugars, artificial sweetening agents, powdered celluloses, such as crystalline and microcrystalline celluloses, flours, gelatins, gums, etc. In the case of tablets for oral use, carriers which are commonly used include lactose and corn starch. Lubricating agents, such as magnesium stearate, can also be added. For oral administration in a capsule form, useful diluents include lactose and dried corn starch. When aqueous suspensions and/or emulsions are administered orally, the compound of the present disclosure may be suspended or dissolved in an oily phase is combined with emulsifying and/or suspending agents. If desired, certain sweetening and/or flavoring and/or coloring agents may be added.

A pharmaceutical composition can be in the form of a tablet. The tablet can comprise a unit dosage of a compound of the present disclosure together with an inert diluent or carrier such as a sugar or sugar alcohol, for example lactose, sucrose, sorbitol or mannitol. The tablet can further comprise a non-sugar derived diluent such as sodium carbonate, calcium phosphate, calcium carbonate, or a cellulose or derivative thereof such as methyl cellulose, ethyl cellulose, hydroxypropyl methyl cellulose, and starches such as corn starch. The tablet can further comprise binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures.

The tablet can be a coated tablet. The coating can be a protective film coating (e.g. a wax or varnish) or a coating designed to control the release of the active agent, for example a delayed release (release of the active after a predetermined lag time following ingestion) or release at a particular location in the gastrointestinal tract. The latter can be achieved, for example, using enteric film coatings such as those sold under the brand name Eudragit®.

Tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Preferred surface modifying agents include nonionic and anionic surface modifying agents. Representative examples of surface modifying agents include, but are not limited to, poloxamer 188, benzalkonium chloride, calcium stearate, cetostearyl alcohol, cctomacrogol emulsifying wax, sorbitan esters, colloidal silicon dioxide, phosphates, sodium dodecylsulfate, magnesium aluminum silicate, and triethanolamine.

A pharmaceutical composition can be in the form of a hard or soft gelatin capsule. In accordance with this formulation, the compound of the present disclosure may be in a solid, semi-solid, or liquid form.

A pharmaceutical composition can be in the form of a sterile aqueous solution or dispersion suitable for parenteral administration. The term parenteral as used herein includes subcutaneous, intracutaneous, intravenous, intramuscular, intra-articular, intraarterial, intrasynovial, intrasternal, intrathecal, intralesional and intracranial injection or infusion techniques.

A pharmaceutical composition can be in the form of a sterile aqueous solution or dispersion suitable for administration by either direct injection or by addition to sterile infusion fluids for intravenous infusion, and comprises a solvent or dispersion medium containing, water, ethanol, a polyol (e.g., glycerol, propylene glycol and liquid polyethylene glycol), suitable mixtures thereof, or one or more vegetable oils. Solutions or suspensions of the compound of the present disclosure as a free base or pharmacologically acceptable salt can be prepared in water suitably mixed with a surfactant. Examples of suitable surfactants are given below. Dispersions can also be prepared, for example, in glycerol, liquid polyethylene glycols and mixtures of the same in oils.

The pharmaceutical compositions for use in the methods of the present disclosure can further comprise one or more additives in addition to any carrier or diluent (such as lactose or mannitol) that is present in the formulation. The one or more additives can comprise or consist of one or more surfactants. Surfactants typically have one or more long aliphatic chains such as fatty acids which enables them to insert directly into the lipid structures of cells to enhance drug penetration and absorption. An empirical parameter commonly used to characterize the relative hydrophilicity and hydrophobicity of surfactants is the hydrophilic-lipophilic balance (“HLB” value). Surfactants with lower HLB values are more hydrophobic, and have greater solubility in oils, while surfactants with higher HLB values are more hydrophilic, and have greater solubility in aqueous solutions. Thus, hydrophilic surfactants are generally considered to be those compounds having an HLB value greater than about 10, and hydrophobic surfactants are generally those having an HLB value less than about 10. However, these HLB values are merely a guide since for many surfactants, the HLB values can differ by as much as about 8 HLB units, depending upon the empirical method chosen to determine the HLB value.

Among the surfactants for use in the compositions of the disclosure are polyethylene glycol (PEG)-fatty acids and PEG-fatty acid mono and diesters, PEG glycerol esters, alcohol-oil transesterification products, polyglyceryl fatty acids, propylene glycol fatty acid esters, sterol and sterol derivatives, polyethylene glycol sorbitan fatty acid esters, polyethylene glycol alkyl ethers, sugar and its derivatives, polyethylene glycol alkyl phenols, polyoxyethylene-polyoxypropylene (POE-POP) block copolymers, sorbitan fatty acid esters, ionic surfactants, fat-soluble vitamins and their salts, water-soluble vitamins and their amphiphilic derivatives, amino acids and their salts, and organic acids and their esters and anhydrides.

The present disclosure also provides packaging and kits comprising pharmaceutical compositions for use in the methods of the present disclosure. The kit can comprise one or more containers selected from the group consisting of a bottle, a vial, an ampoule, a blister pack, and a syringe. The kit can further include one or more of instructions for use in treating and/or preventing a disease, condition or disorder of the present disclosure, one or more syringes, one or more applicators, or a sterile solution suitable for reconstituting a pharmaceutical composition of the present disclosure.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

### EXAMPLES

### Example 1: TFEB Confers Sensitivity to Apilimod

We have previously shown that apilimod is a highly cytotoxic agent in TSC null cells. In these cells, the mTOR pathway is constitutively active, as it is in a number of cancers. A screen of over 100 cancer cell lines showed that apilimod was cytotoxic in diverse types of cancers. Our results further indicated that the cytotoxic activity of apilimod was due to inhibition of intracellular trafficking and a corresponding increase in apoptosis and/or autophagy.

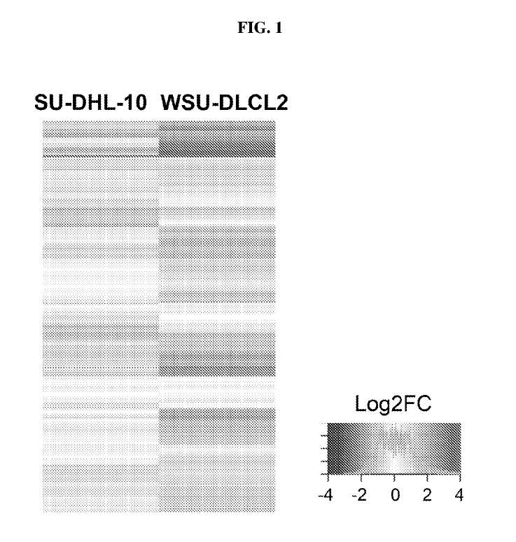

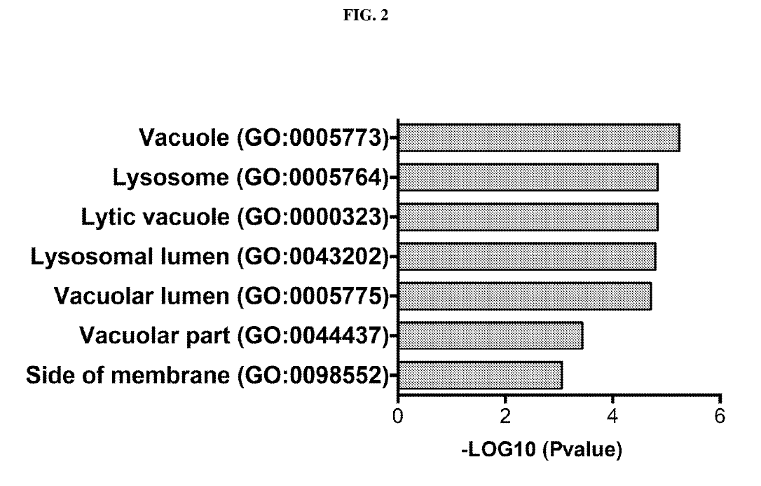

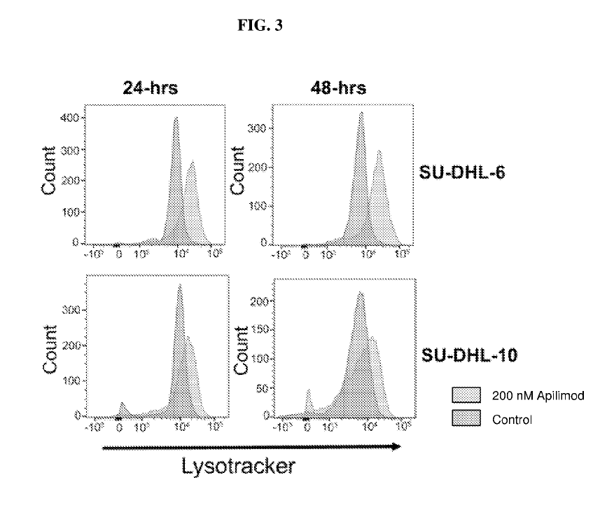

To further understand apilimod's cellular mechanism of action, we performed a global gene expression analysis of the changes occurring after apilimod treatment of two non-Hodgkins B cell lymphoma (B-NHL) tumor cell lines, SU-DHL-10 and WSU-DLCL2. FIG. 1 shows a heatmap representation of the gene expression changes. Up-regulated genes are represented by red and are clustered toward the top of the heatmap while down-regulated genes are represented by blue and cluster towards the bottom. We next performed a gene ontology analysis. As shown in FIG. 2, there was a clear enrichment of lysosomal genes induced by apilimod. We next used LysoTracker staining to examine the acidified compartment of the cells. FIG. 3 shows that there was an expansion of the acidified compartment, suggesting that lysosomal biogenesis is up-regulated by apilimod, consistent with the gene expression findings.

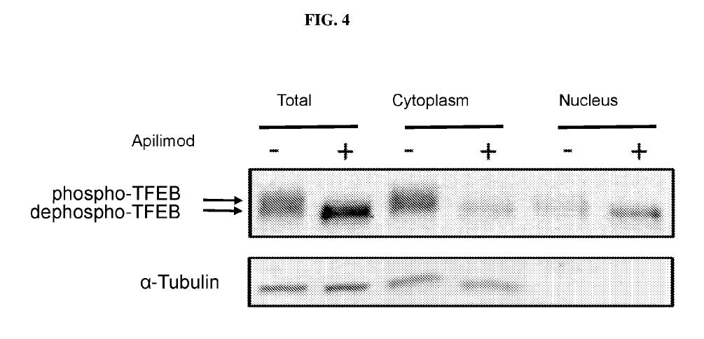

TFEB is a master regulator of lysosomal gene expression and is activated by dephosphorylation followed by nuclear translocation (Roczniak-Ferguson et al., 2012; Settembre et al., 2012). Accordingly, we next looked at TFEB phosphorylation status and subcellular localization following apilimod treatment. FIG. 4 shows that within two hours of treatment, TFEB is dephosphorylated (indicated by an increased electrophoretic mobility) and translocates into the nucleus, as has been observed in other cell types (Wang et al., 2015). Collectively, these results indicated that apilimod induces TFEB dephosphorylation and nuclear translocation, followed by enhanced lysosomal gene expression.

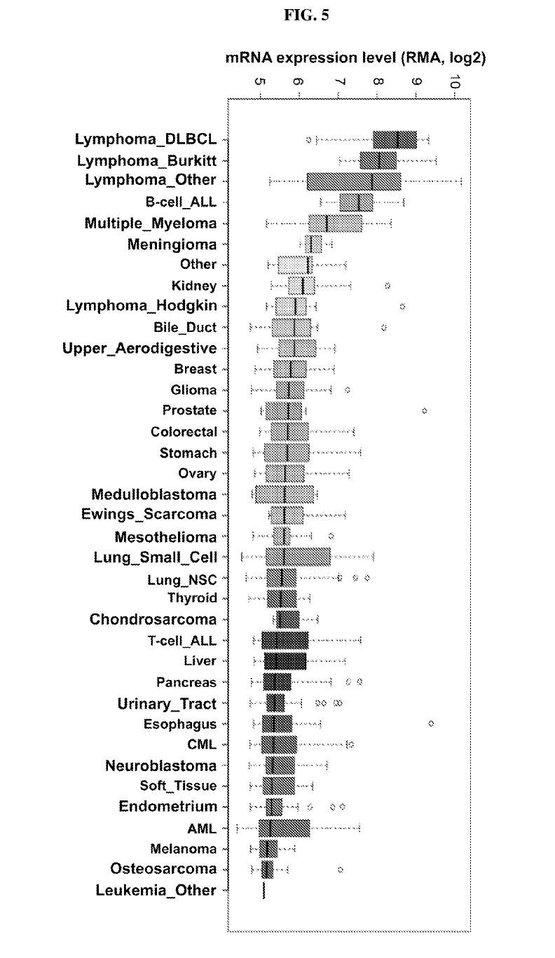

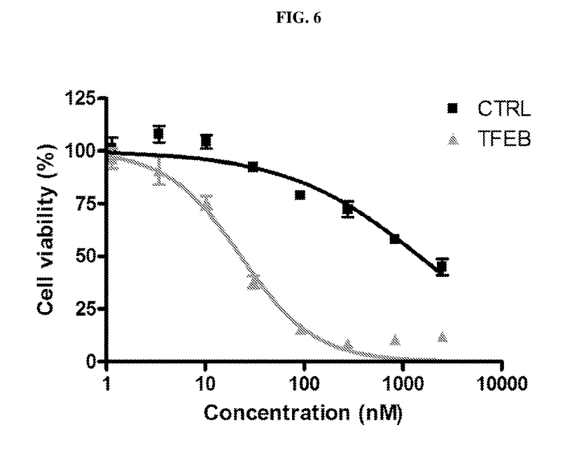

To further explore the role of TFEB in the cellular response to apilimod, the expression levels of TFEB across different cancer cell lines from the Cancer Cell Line Encyclopedia (CCLE) database (Barretina et al., 2012) were extracted and examined to determine whether there was a correlation between TFEB expression and the sensitivity of the cell line to apilimod. FIG. 5 shows the expression levels of TFEB in a number of cell lines. As shown in FIG. 5, TFEB is highly expressed in B-NHL compared to all other tumor types. To test whether the increased TFEB expression contributes to the apilimod sensitivity observed in B-NHL, we overexpressed TFEB in the TFEB-deficient B-NHL cell line CA46. As shown in FIG. 6, TFEB over-expression enhanced apilimod sensitivity by greater than 50-fold.

In summary, the data presented here demonstrates that high levels of TFEB expression confer sensitivity to apilimod.

### Example 2: Microphthalmia (MiT) Transcription Factors in Cancer

TFEB is a member of the MiT family of basic helix-loop-helix leucine zipper transcription factors, a family which also includes the highly homologous genes MITF, TFE3, and TFEC (Fisher et al. 1991). MITF is the most well characterized member of this family and is a master regulator of melanocyte biogenesis as well as an established driver of melanoma and clear cell sarcoma (Steingrimsson et al. 2004; Garraway et al. 2005; Davis et al. 2006). TFEB and TFE3 overexpression through chromosomal translocation are implicated in renal cell carcinogenesis, and TFE3 overexpression is further implicated in alveolar soft part sarcoma and perivascular epitheloid cell tumors (reviewed in Haq et al. 2011; Kauffman et al. 2014). TFEC expression is limited to macrophages, and is the least studied MiT family member, with little known about its function (Rehli et al. 1999). Members of the MiT family are known to regulate endolysosomal and autophagosome biogenesis as well as autophagy, with TFEB in particular noted as a master regulator of lysosomes (Sardiello et al. 2009; Settembre et al. 2012; Martina et al. 2014; Ploper et al. 2015). Given that high levels of TFEB confers sensitivity to apilimod in B-NHL, we propose that other cancers characterized by increased expression and/or activity of one or more of the MiT family of transcription factors will also be sensitive to apilimod. These may include, but are not limited to, renal cell carcinoma, melanoma, clear cell sarcoma, alveolar soft part sarcoma, and perivascular epitheloid cell tumors.

### Example 3: Renal Cell Carcinoma

TFEB, TFE3 and MITF have each been implicated in renal carcinogenesis by altering kidney cell metabolism in concert with other genes frequently mutated in renal cell carcinomas (RCCs) such as VHL, MET, FLCN, fumarate hydratase, succinate dehydrogenase, TSC1 and TSC2 (Linehan 2012). TFEB and TFE3 translocations resulting in overexpression and nuclear localization of functional transcription factor occur in translocation carcinomas, a rare RCC subtype accounting for 1-5% of sporadic RCCs (Shuch et al. 2015). Furthermore, a novel oncogenic MITF fusion gene, ACTG1-MITF was identified RCC (Durinck et al, 2015). Finally, germline missense mutations of MITF resulting in increased transcriptional activity have been reported in both melanomas and RCCs (Bertolotto et al. 2011).

RCCs are the most common type of kidney cancer in adults, accounting for 2-3% of adult malignancies and 90-95% of neoplasms arising from the kidney (Cancer Facts and Figures 2015). There are approximately 65,000 new cases of RCC in the United States each year and about 13,500 deaths from RCC annually, with 65 as the median age at onset of the disease (Siegel et al. 2012). Advanced RCCs are treated with nephrectomies combined with targeted therapies that include VEGF inhibitors (Sunitinib, Pazopanib, Bevacizumab, Sorafenib, Cabozantinb and Axitinib) and mTOR inhibitors (Everolimus and Temsirolimus) (Cancer Facts and Figures 2015).

Clear cell RCC is the predominant histological subtype of RCC, accounting for approximately 75% of all RCCs (Shuch et al. 2015). These tumors are named for their abundant clear cytoplasm due to lipid and glycogen deposition (Shuch et al. 2015). Clear cell RCC tends to present with higher grade, metastatic disease with poor prognosis (Shuch et al. 2015). Mutations in the von Hippel-Lindau gene VT-IL are reported to occur in up to 90% of clear cell RCC (Nickerson et al. 2008). Aberrations in VHL function as a ubiquitin ligase result in accumulation of the transcription factor HIF-alpha and upregulation of hypoxia-responsive genes, including VEGF and PDGF, and subsequent induction of signaling pathways such as RAF-MEK-ERK and PI3K-Akt-mTOR (Clarke 2009).

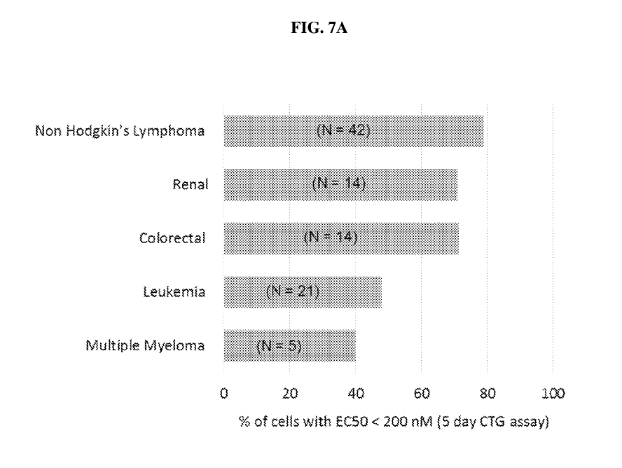

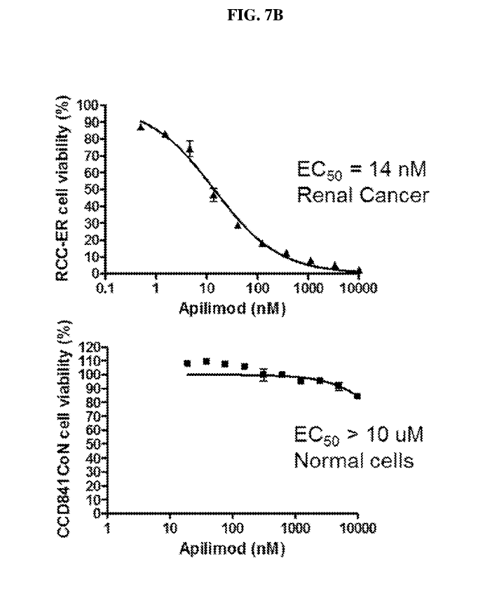

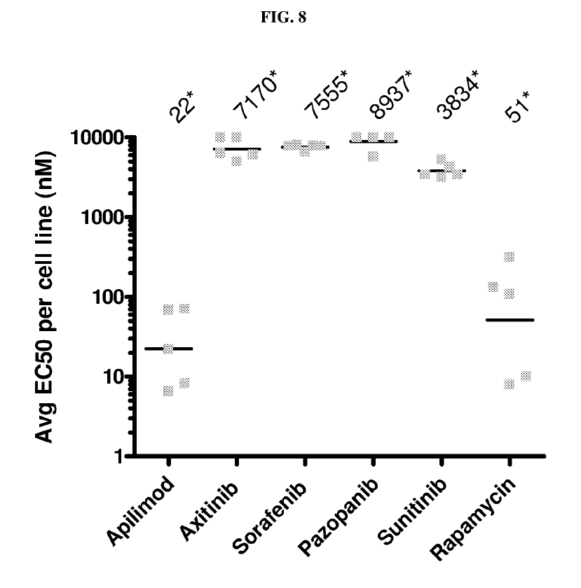

We next demonstrated that numerous clear cell RCCs cell lines display in vitro sensitivity to apilimod, with sensitivity defined as an EC50 less than 200 nM in a 5 day assay (FIG. 7 and Table 1 below). We also showed that apilimod has superior activity against clear cell RCCs relative to the standard of care treatments, axitinib, sorafenib, pazopanib, sunitinib, and rapamycin in 5 day assays (FIG. 8).

TABLE 1
Average EC50 values from 10 clear cell RCC lines
tested with apilimod in a 5 day assay.

| | EC50 (nM) |
|---|---|
| Cell Line | (avg, n = 2) |
| 769-P | 44 |
| RCC-MF | 8 |
| RCC-ER | 9 |
| RCC-FG2 | 32 |
| RCC-JF | 60 |
| 786-0 | 71 |
| A-704 | 11 |
| RCC-JW | 27 |
| KMRC-1 | 4 |
| KMRC-3 | 39 |

Our data also suggest that apilimod is preferentially cytotoxic in RCC having a VHL mutation, as shown in Table 2.

TABLE 2
Average EC50 values from 12 clear cell RCC lines
tested with apilimod in a 5 day assay and VHL status.

| | EC50 (nM) | |
|---|---|---|
| Cell Line | (avg, n = 2) | VHL status |
| Caki-1 | 2276 | WT |
| ACHN | 9039 | WT |
| 769-P | 44 | mutant |
| RCC-ER | 9 | mutant |
| RCC-FG2 | 32 | mutant |
| RCC-JF | 60 | mutant |
| 786-0 | 71 | mutant |
| A-704 | 11 | mutant |
| KMRC-1 | 4 | mutant |
| KMRC-3 | 39 | mutant |
| Caki-2 | 3355 | mutant |
| A498 | 84009 | mutant |

Given that clear cell RCCs display sensitivity to apilimod, we propose that other RCC subtypes will also be sensitive to apilimod, including but not limited to papillary type I and type II, chromophobe, hybrid, oncocytoma, translocation, angiomyolipoma, oncocytic, medullary, and collecting duct carcinomas.

### Example 4: TFEB Translocation Renal Cell Carcinomas

TFEB translocation RCCs are rare, with approximately 50 cases reported to date (Argani 2015). This number may be underrepresented due to misdiagnosis as a result of a lack of established clinical characteristics. Of the 50 cases, 4 have involved metastases, resulting in death in 3 of these cases (Argani, 2015). The median age of onset of TFEB translocation RCCs is 31 years, with childhood cytotoxic chemotherapy implicated as a cause in a subset of cases (Argani, 2015).

TFEB translocation RCCs harbor a specific t(6;11) (p21; q12) translocation that fuses the TFEB locus to Alpha (MALAT1), a noncoding RNA of unknown function, resulting in the overexpression of full length TFEB (Davis et al. 2003; Kuiper et al. 2003; reviewed in Kauffman et al. 2014). The Alpha-TFEB fusion gene may be detected by RT-PCR, DNA PCR, or FISH (Argani et al. 2005; Argani et al. 2012). Furthermore a CLCTC-TFEB fusion gene was recently identified in a translocation RCC tumor which is predicted to function like the Alpha-TFEB fusion gene. (Durinck et al., 2015). TFEB translocation RCCs exhibit 30-60 fold upregulation of TFEB transcript by qRT-PCR and this results in high expression of TFEB protein relative to normal kidney tissues as assessed by Western blot (Kuiper et al. 2003) and strong nuclear TFEB staining detected by IHC (Argani et al. 2005).

Clinically, TFEB translocation RCCs do not have a distinctive appearance and can be given the broad differential diagnosis of high grade unclassified RCC (Argani, 2015). However, these tumors demonstrate nuclear TFEB immunoreactivity by IHC, and also stain positive for the melanoma markers Melan A and HMB45 along with the cysteine protease cathepsin K (Argani et al. 2005; Martignoni et al. 2009). As the TFEB breakapart FISH assay is less affected by variable fixation, it is the preferred diagnostic test for TFEB translocation RCC in formalin-fixed and paraffin-embedded material (Argani et al. 2012).

Although the clinical implication of TFEB translocation RCC has been established, there are currently no effective therapeutics for advanced translocation RCC. Given we have demonstrated that high levels of TFEB confers sensitivity to apilimod in B-NHL, the supposition is that TFEB translocation RCCs will also demonstrate exquisite sensitivity to apilimod.

### Example 5: TFE3 Translocation Renal Cell Carcinomas

TFE3 translocation RCCs account for 40% of pediatric RCCs and less than 5% of adult RCCs, resulting in approximately 10 new pediatric and 1.260 new adult cases in the U.S. each year (Magers et al. 2015). As in TFEB translocation RCCs, childhood cytotoxic chemotherapy is implicated as a cause of TFE3 translocation RCC, which tends to occur 2-14 years after exposure. Pediatric cases of TFE3 RCC tend to be indolent, however adult cases frequently involve early nodal involvement, aggressive metastases and a prognosis similar to that of clear cell RCC (Magers et al. 2015, Geller et al. 2008). Furthermore, these tumors can reappear as untractable metastatic tumors decades after complete tumor resection (Dal Cin P et al, 1998; Rais-Bhahrami S, et al, 2007).

Clinically, TFE3 translocation RCCs resemble clear cell RCCs, with papillary architecture and epithelioid clear cells. Morphology can vary to resemble other RCC subtypes, making classification difficult (Argani, 2015). Of pertinence, these tumors demonstrate strong nuclear TFE3 immunoreactivity by IHC, and also stain positive for CD10 and RCC antigen, and frequently stain positive for cathepsin K (Argani et al. 2003; Argani et al. 2005; Martignoni et al. 2009).

Five type of translocations involving the TFE3 locus on Xp11 have been documented in the literature to date: PRCC-TFE3, ASPSCR1-TFE3, SFPQ-TFE3, NONO-TFE3, and CLTC-TFE3 (Kauffman et al. 2014). PRCC-TFE3, ASPSCR1-TFE3, and SFPQ-TFE3 translocations have been confirmed as recurrent mutations identified in multiple patients, while NONO-TFE3 and CLTC-TFE3 fusion genes have been identified in single patients to date (Kauffman et al. 2014). Notably, the ASPSCR1-TFE3 fusion gene has also been identified as a recurrent mutation in alveolar soft part sarcomas, a rare lung cancer type, and the SEPQ-TFE3 fusion gene has been identified in perivascular epithelioid cell neoplasms (Landanyi et al. 2001; Tanaka et al. 2009). The breakpoint sites of these fusion genes varies, but result in a fusion product with the N terminal portion of each fusion partner linked to a range of the C-terminal TFE3 exons. All fusion partners have constitutively active promoters, resulting in the overexpression of functional TFE3 protein (Kauffman et al. 2014). The TFE3 translocation may be detected by strong nuclear staining with an antibody against the TFE3 C-terminus or by break apart FISH assay (Argani 2015).

Several cell lines have been derived from TFE3 translocation RCCs; the PRCC-TFE3 fusion gene is present in cell lines UOK120. UOK124, and UOK146; the SFPQ-TFE3 fusion gene is present in the UOK145 cell line; the ASPSCR1-TFE3 fusion gene is present in the FU-UR1 cell line; and the NONO-TFE3 fusion gene is present in the UOK109 cell line (Kauffman et al. 2014).

We believe it is likely that TFE3 translocation cell lines will also demonstrate sensitivity to apilimod, including but not limited to TFE3 translocation RCCs, alveolar soft part sarcoma and perivascular epitheloid cell neoplasms.

### Alveolar Soft Part Sarcoma

Alveolar soft part sarcoma (ASPS) is a rare slow growing neoplasm with an unknown cell of origin. ASPS represents less than 1% of the 12,000 new cases of soft tissue sarcomas diagnosed per year in the U.S. (Jaber and Kirby 2014; National Cancer Institute 2014) and tends to involve the soft tissues of the thighs or buttocks in adults and head and neck region in children (Jaber and Kirby 2014). Metastases to the lung, hone, brain and/or liver occur in up to 79% of patients (Lieberman et al. 1989; Portera et al. 2001). Metastatic ASPS is usually resistant to conventional radiation and chemotherapy (Jaber and Kirby 2014).

ASPS is characterized by the specific translocation der(17)t(X;17)(p11;25), which fuses TFE3 to ASPSCR1, resulting in overexpression and nuclear localization of functional TFE3 (Jaber and Kirby 2014). Cells within the tumor have a distinctive organoid, alveolar-like pattern of growth with cells containing periodic acid-Schiff, distase-resistant intracytoplasmic crystals (Jaber and Kirby 2014). Tumors are consistently positive for strong nuclear TFE3 staining by IHC, as well as for cathepsin K. To date only one cell line has been derived from an ASPS tumor, the cell line ASPS-1 (Kenney et al. 2011).

Given that TFEB overexpression confers sensitivity to apilimod, we believe that cancers and cell lines containing overexpression of other MITF family members such as TFE3 will also be sensitive to apilimod, including but not limited to ASPS and the cell line ASPS-1.

### Birt-Hogg-Dubé Syndrome

Birt-Hogg-Dubé (BHD) syndrome is an autosomal dominant genetic disease (Birt et al., 1977) arising from mutations on chromosome 17 (17p11.2) in the folliculin (FLCN) gene (Schmidt et al, 2005), the only known susceptibility gene for BHD. FCLN BHD mutations are diverse with 53 unique germline mutations identified (Reviewed in Wei et al. 2009). Patients with BHD are predisposed to a variety of proliferative diseases including follicular hamartomas, lung cysts and kidney neoplasms (BHDsyndrome.org).

Mechanistically, FLCN complexes with folliculin-interacting protein-1 (FNIP1) and -2 (FNIP2) and 5′-AMP-activated protein kinase (AMPK), connecting it to regulation of mTOR and thereby regulation of cellular proliferation (Baba et al 2010). In addition, by modulating FLCN expression in UOK257 cells, a clear-cell renal tumor cell line from a BHD patient in which FLCN is mutated (Yang et al), FLCN was shown to negatively regulate TFE3 nuclear localization (Hong et al 2010). Consistent with this finding, ARPE-19 cells depleted of FLCN showed TFE3 nuclear accumulation (Martina et al. 2014). Analysis of UOK257 xenografts showed tumors stained positive for nuclear TFE3, while normal adjacent kidney tissue displayed weak cytoplasmic staining. Further corroborating these findings, Flcn-null MEFs displayed TFE3 localized in the nucleus. Finally, IHC staining in tumor samples from BHD patients revealed nuclear or nuclear/cytoplasmic staining. To confirm FLCN inactivation resulted in increased TFE3 transcriptional activity, the TFE3 target GPNMB was used as a surrogate marker and was shown to be increased in tumors from BHD patients relative to normal kidney tissues via western blot analysis. IHC further verified GPNMB expression in tumor but not normal tissue in sections from BHD patients and Flcn+/− heterozygote mouse renal tumors (Hong et al 2010). Collectively, these findings link functional inactivation of FLCN, as observed in patients with BHD, with in increased nuclear TFE3 localization and transcriptional activity.

Given our supposition that apilimod will be effective in tumors with high levels of nuclear TFE3, tumors with mutations in Flcn which include, but not limited to, renal cancer (Paulovich et al 2002), colorectal cancer (Kahnoski et al 2003), endometrial cancer (Fujii et al 2006), gastric cancer (Jiang et al 2007), are also expected to be sensitive to apilimod.

1-41. (canceled)

42. A method for treating cancer in a subject in need thereof, the method comprising
assaying a sample of cancer cells from the subject for the overexpression of a microphthalmia (MiT) transcription factor, and
treating the subject having cancer cells overexpressing an MiT transcription factor with a pharmaceutical composition comprising apilimod, or a pharmaceutically acceptable salt thereof.

43. The method of claim 42, wherein the pharmaceutically acceptable salt of apilimod is apilimod dimesylate.

44. The method of claim 42, wherein the MiT transcription factor is selected from the group consisting of TFEB, TFE3, TFEC, and MITF.

45. The method of claim 44, wherein the MiT transcription factor is TFEB or TFE3.

46. The method of claim 42, wherein the cancer is a non-Hodgkins B cell lymphoma, a renal cell carcinoma, a melanoma, a thyroid carcinoma, a clear cell sarcoma, an alveolar soft part sarcoma, or a perivascular epitheloid cell tumor.

47. The method of claim 46, wherein the cancer is a renal cell carcinoma.

48. The method of claim 47, wherein the renal cell carcinoma contains a TFEB translocation.

49. The method of claim 48, wherein the TFEB translocation is a t(6;11) (p21; q12) translocation.

50. The method of claim 47, wherein the renal cell carcinoma is selected from the group consisting of a papillary type I or type II, a chromophobe, a hybrid, an oncocytoma, a translocation, an angiomyolipoma, an oncocytic, a medullary, and a collecting duct carcinoma.

51. The method of claim 47, wherein the renal cell carcinoma is selected from clear cell renal carcinoma, a transitional cell carcinoma, Wilms tumor (nephroblastoma), renal sarcoma, and benign (non-cancerous) kidney tumors, renal adenoma, oncocytoma, and angiomyolipomas.

52. The method of claim 47, wherein the renal cancer has a mutation in the von Hippel-Lindau (VHL) gene.

53. The method of claim 42, wherein the method further comprises administering to the subject at least one additional active agent selected from the group consisting of a protein kinase inhibitor, a PD-1/PDL-1 pathway inhibitor, a checkpoint inhibitor, a platinum based anti-neoplastic agent, a topoisomerase inhibitor, a nucleoside metabolic inhibitor, an alkylating agent, an intercalating agent, a tubulin binding agent, and combinations thereof.

54. The method of claim 53, wherein the at least one additional active agent is a vascular endothelial cell growth factor (VEGF) inhibitor.

55. The method of claim 54, wherein the VEGF inhibitor is selected from the group consisting of sunitinib, pazopanib, bevacizumab, sorafenib, cabozantinb and axitinib.

56. The method of claim 53, wherein the at least one additional active agent is pazopanib or sorafenib, or a combination thereof.

57. The method of claim 53, wherein the at least one additional active agent is a PD-1/PDL-1 pathway inhibitor.

58. The method of claim 53, wherein the at least one additional active agent is selected from pembrolizumab (Keytruda), avelumab, atezolizumab (MPDL3280A), nivolumab (BMS-936558), pidilizumab (CT-011), MSB0010718C, and MEDI4736.

59. The method of claim 42, wherein the cancer is refractory to standard treatment or is metastatic.

60. A method for treating cancer in a subject having cancer cells overexpressing one or more microphthalmia (MiT) transcription factor, the method comprising administering to the subject a therapeutically effective amount of a composition comprising apilimod, or a pharmaceutically acceptable salt thereof.

61. The method of claim 60, further comprising a pretreatment step of assaying a sample of the cancer cells for the overexpression of one or more MiT transcription factors.

62. The method of claim 61, wherein the assaying comprises detection of one or more of TFEB, TFE3, TFEC, and MITF.
